(19) United States
(12) Patent Application Publication
(10) Pub. No.: US 2015/0132788 A1
(43) Pub. Date: May 14, 2015

(54) ESTABLISHING THE VIABILITY OF BIOLOGICAL SAMPLES

(71) Applicant: Ovamed GmbH, Barsbüttel (DE)

(72) Inventors: Detlev Goj, Hamburg (DE); Ulf-Georg Bickmeyer, Bremersbek (DE); Guido Schramm, Ammersbek (DE)

(73) Assignee: Ovamed GmbH, Barsbüttel (DE)

(21) Appl. No.: 14/408,229

(22) PCT Filed: Jun. 11, 2013
(86) PCT No.: PCT/EP2013/061955

§ 371 Date: Dec. 15, 2014

(30) Foreign Application Priority Data
Jun. 15, 2012 (EP) 10 2012 210 055.5

(51) Int. Cl. G01N 33/84; G01N 33/50; G01N 33/58

(52) U.S. Cl. CPC G01N 33/84; G01N 33/582; G01N 33/5091
USPC 435/29; 546/118; 435/288.4; 435/288.7

(57) ABSTRACT

The invention relates to a method, a use and a kit for establishing the viability (ability to live) of biological samples using ageladine A. The invention is advantageously suitable for establishing the viability of biological samples which can otherwise be dyed in an intra-cellular manner by fluorescence dyes other than ageladine A not at all or only with difficulty. The invention is found to be very particularly advantageous for establishing the viability of eggs (ovaries) of the pig whipworm which are intended to be used for the treatment or prophylaxis of specific (gastroenterological) autoimmune diseases.

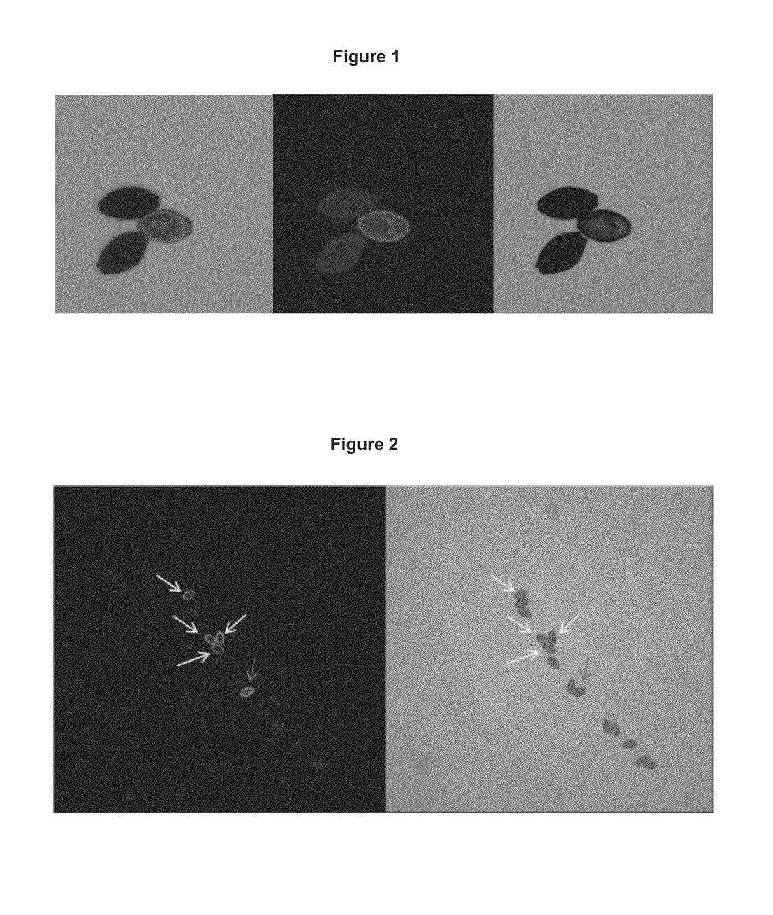

The invention relates to a method, a use and a kit for establishing the viability (ability to live) of biological samples.

Methods for assessing the biological viability (ability to live) are known in the prior art such as, for example, for the cell viability (cell ability to live, number of living cells) which denotes in microbiology the proportion of living calls in a cell population. Establishing viability is based on properties of living cells such as endocytosis, enzyme activity, intactness of the cell membrane or replication. Since each method has weaknesses, sometimes fluorescent double-dyes are carried out for simultaneously establishing apoptosis and necrosis, such as, for example, vital fluorescence double-dyeing with propidium iodide (PI) and annexin V (see below). Double-dyed cells with annexin V and PI indicate dead cells whereas simply PI-dyed cells are classified as necrotic and simply annexin-V-dyed cells are classified as apoptotic. The methods for establishing, the biological viability (ability to live) therefore identify either living cells or dead cells.

The term “living” is intended to refer in this instance to properties of living beings, which properties are defined in biology and delimit them as “living” from non-living and dead systems in nature. All the characteristics must apply to all living organisms (“living beings”) at least at the cell level. Dead organisms had all the characteristics in the past. Organisms which do not have all the characteristics and are therefore similar to dead organisms or non-living objects but which can become living organisms at any time have latent life, for example, spores of bacteria or fungi. Non-living objects do not exhibit all the characteristics at the time of their existence. In addition, there are hypothetical early stages of the evolution of life and recent borderline forms of life such as, for example, viruses. Three significant properties have been found, however, and are supposed to apply to all living beings in the context of this text as definition criteria: 1) metabolism during at least one life phase which requires compartmentalisation by a wall or membrane; 2) capacity for self-reproduction; and 3) the genetic variability connected with self reproduction as a condition of evolutionary development. Consequently, living beings are defined in biology as organised, genetic units which are capable of metabolism, propagation and evolution, that is to say, they comply with the criteria of the living being.

The above-mentioned methods for establishing the biological viability (ability to live) which identify either living cells or dead cells cannot currently be applied in specific cases so that, in the prior art use is still made of in-vivo methods, as is the case in medically relevant parasite systems. For example, microscopically small eggs (ova) of the parasite Trichuris suis (pig whipworm) which belongs to the genus of helminthes are produced for medical purposes. Those eggs are obtained from pigs in an impure and non-embryonated state, cleaned under controlled conditions and brought to embryonation. In specific quantities (doses) and in a phosphate-buffered saline solution, those eggs are used as a medicament for treating autoimmune illnesses. This use in human medicine is based on studies of the University of Iowa (Iowa City, USA) and the ingestion of the eggs of the pig whipworm (Trichuris suis ova) has a positive effect in humans on the remission or recidivism prophylaxis in patients with chronically inflammatory intestine diseases. The therapeutic approach is based on the assumption that the immune system which over-acts in auto-Immune diseases would no longer attack the intestine wall if it is given a different task. This therapy, for example, for patients with Morbus Crohn and Colitis ulcerosa, is also known by the name TSO, where TSO stands for Trichuris Suis Ova, the eggs of the pig whipworm (Trichuris suis). Consequently, ISO is a therapeutic agent which contains the eggs of the pig whipworm, which eggs cannot be seen with the naked eye and which measure only roughly a 20th of a millimetre. Each dose of the therapeutic agent which is produced as a recipe medicament contains 2500 eggs floating in fluid. The eggs of the pig whipworm are harmless to humans because they only survive for a short time in humans as unnatural hosts and also cannot multiply therein any more, and in addition after ingestion no symptoms such as stomach pains occur. The eggs are swallowed—in a sterilised state but not killed—in a neutral suspension and then accumulate in the region of the duodenum. They, survive at that location for approximately 14 days, then become detached from the intestine wall and are digested. It may sporadically be the case that occasionally a small worm hatches. However, it immediately dies and atrophies because it is in the incorrect host.

The manner in which the eggs of Trichuris suis act in the intestine in bio-immune therapy with ISO in Crohn and colitis patients has not yet been definitively explained. However, it is assumed that the eggs stimulate the immune system in such a manner that the typical disruptions for Morbus Crohn und Colitis ulcerosa are compensated for in the immune system. For patients with intestine inflammation, for example, an overactives immune response of the T helper cells of the type 1 (TH-1 cells) is typical, possibly a pathological reaction to substances which are physiologically located in the intestine lumen. However, worm eggs regulate the inflammation-inhibiting TH-2 cell system in terms of magnitude. Consequently, the viability (ability to live) of the eggs of the pig whipworm is decisive for the quality and efficacy of the therapeutic agent and must be established during the production and acquisition process. The culture of the pig whipworm is, however, complex. The whipworms have to mature for three months in practically sterile domestic pigs. Then, the excretions of the animals are collected and the worms which are now pregnant (embryonated) can be separately obtained. A pig thus provides approximately 1 million worm eggs. 2500 eggs are required for an immunologically effective single dose. Before the eggs of the pig whipworm can be released as a medicament by the head of production to produce immunologically effective single doses, therefore, samples from a homogeneous production batch have to be examined in terms of their ability to live (viability). This is currently carried out in the prior art with a complex method in which the samples are brought back into the pig as the natural host of that parasite. There, new worms which again produce eggs and therefore demonstrate their viability, then develop from the eggs in the intestine of the pig within approximately three months. According to legal provisions, animals which were part of a production process for medicaments are killed after their use has been achieved, whereby that viability test is not only cost-intensive and time-intensive but also ethically questionable.

Therefore, there was the objective of providing an economical viability test which is as simple and reliable as possible in technical terms, in particular as a generally applicable in-vitro method for assessing the viability (ability to live) of biological samples, which method overcomes the disadvantages of the prior art. The object of the present invention also particularly involved providing a suitable method, a suitable use and a suitable kit for establishing the viability (ability to live) of biological samples, which method allows the use of fluorescence dyes and is also suitable for replacing in-vivo methods of the prior art.

In the context of the present invention, it has now been found that degenerated eggs of the Trichuris suis (pig whipworm) have within their membrane a low pH value but viable eggs, that is to say, eggs capable of living, have a high pH value. Therefore, the present invention proposes in a particularly preferred embodiment that the viability of the eggs of the pig whipworm be replaced by the use of ageladine A in place of the complex in-vivo reinfection test in pigs. The tests according to the invention for dyeing the Trichuris suis eggs with the pH-sensitive vital dye ageladine A were successfully carried out and demonstrated that ageladine A fluoresces under ultraviolet excitation by means of multi-photon microscopy in degenerated eggs of Trichuris suis. As a result, the method allows a differentiation between viable and non-viable nematode eggs and is further generally suitable for in-vitro methods for assessing the viability (ability to live) of a large number of biological samples.

Accordingly, the invention relates to a method for assessing the ability to live (viability) of one or more biological samples comprising a) the provision of the biological samplers) to be examined; b) the incubation of the biological sample(s) provided at a) with ageladine A; c) the ultraviolet excitation of the incubated biological sample obtained at b) with UV light of a wavelength of 370 nm; and d) the detection of fluorescing and non-fluorescing portions of the biological sampler(s) excited with ultraviolet according to c) in the range between 410 and 480 nm.

The pyrrole/imidasole alkaloid ageladine A is a natural substance from the sea sponge Agelas clathrodes, also known by the name elephant ear sponge, and can now also be obtained with a synthetic method. Ageladine A exhibits fluorescence in the blue/green range in accordance with the pH value. It is bromated and facilitates membrane penetration in cell materials, whereby non-damaging dyeing of the cells becomes possible. The greatest fluorescence by ageladine A occurs in the region of pH 4 and decreases as the pH value increases.

DE10 2007 034 886 A1 already describes an optical measuring method for establishing the pH value of a medium by the addition of ageladine A as a fluorescing pH value indicator into the medium, fluorescence excitation of the pH value indicator by irradiating the pH value indicator with light of at least one selected wavelength and detection of the emitted fluorescence intensities of the pH value indicator as a measurement for the pH value of the medium.

DE 10 2007 034 886 A1 describes the use of ageladine only generally as a pH indicator. However, the suitability of ageladine for use as the viability test and pH measurements for the purpose of a viability test is not mentioned in DE 10 2007 034 886 A1. A viability test with pH-sensitive dyes is already known from the prior art from U.S. patent specification U.S. Pat. No. 6,800,675 B2. U.S. Pat. No. 6,800,675 B2 relates in this instance to systems including compositions and methods for measuring the pH value, in particular in cells, organelles and other samples for examining the cell viability, wherein the compositions comprise a pH-sensitive fluorescing agent and fluorogenic 2′,7′-dialkylfluorescein derivatives and associated non-fluorescing precursor compounds. The compositions allow quotient-metric measurements in the excitation spectrum and the emission spectrum. The methods described comprise the addition of a precursor compound to a cell sample, incubation of the cell sample in order to release the free indicator, illumination of the cell sample and establishment of the fluorescence response of the free indicator. However, a viability test using ageladine or for cells, tissue and organisms which otherwise poorly absorb pH indicators and have to be incubated for a relatively long time end consequently require a pH indicator, such as, for example, ageladine which is also non-toxic with a relatively long incubation time, is not described either by DE 10 2007 034886 A1 or by U.S. Pat. No. 6,800,765 B2.

In particular, DE 10 2007 034886 A1 and U.S. Pat. No. 6,800,765 B2. also do not contain any indications of the use according to the invention of ageladine in a viability identification operation, particularly in whipworm eggs in which there is an additional problem, that is to say that the introduction of pH indicators into those eggs is generally difficult and is successful only with ageladine which is not cell-toxic even after a long incubation time.

The suitability of ageladine in a viability identification operation in biological samples, for example, in medically relevant parasite systems, such as particularly in whipworm eggs, In which there is the problem that the introduction of pH indicators is difficult, which therefore may not be able to be dyed at all or can be dyed only to an insufficient extent or only under long and/or intensified incubation conditions, is therefore surprising,

Therefore, the invention also relates to a method according to the invention using ageladine in order to assess the ability to live (viability) of biological samples which is distinguished in that the biological samples can be dyed in an intra-cellular manner by conventional fluorescence dyes other than ageladine A only with intensified, that is to say, for example, not at all or only to an insufficient extent or only under long and/or intensified incubation conditions.

Preferred methods according to the invention using ageladine are methods for assessing the ability to live (viability) of biological samples which are distinguished in that the biological samples resulted from biological preparations which are used in a viable form to produce medicaments and/or therapeutic agents. Such biological preparations may be, for example, medically relevant parasite systems, in particular especially biological preparations from whipworm eggs. However, the invention is not limited to such medically relevant parasite systems but instead generally includes methods for assessing the ability to live (viability) of biological samples which are distinguished in that the biological sample is selected from the group consisting of (lower) organisms, in particular microorganisms, parasitic and non-parasitic organisms, cells, individual cells or cell colonies, tissues, organelles, sperm, egg cells and ovaries. However; the biological sample is preferably selected from the group consisting of parasitic and non-parasitic organisms and ovaries of such parasitic and non-parasitic organisms, in a particularly preferred method according to the invention, the methods are used to assess the ability to live (viability) of biological samples, there being selected as biological samples ovaries of parasitic- and/or non-parasitic worms, preferably ovaries of parasitic and/or non-parasitic worms which are used in a viable form to produce medicaments and/or therapeutic agents, in this instance, particular significance is attributed is methods according to the invention for assessing the ability to live (viability) of biological samples in which there are selected as biological samples ovaries of parasitic worms, preferably ovaries of helminthes, particularly preferably ovaries of the pig whipworm (Trichuris suis).

In a particularly advantageous manner, it is possible to use the method according to the invention in order to assess the ability to live (viability) of biological samples in which a decreasing ability to live (viability) of the biological samples is associated with a change of the intra-cellular pH value, preferably with a reduction of the intra-cellular pH value, and particularly preferably with a reduction of the intra-cellular pH value in the acidic pH range.

According to the invention, ageladine A is used in the above-mentioned methods according to the invention in order to assess the ability to live (viability) of biological samples. Therefore, the present invention also relates to the use of ageladine A in a method or kit in order to assess and/or to establish the ability to live (viability) of one or more biological sample(s) by means of fluorescence, preferably biological samples which can be intracellularly dyed by conventional fluorescence dyes other than ageladine A only with difficulty, that is to say, for example, not at all or only to an insufficient extent or only under long and/or intensified incubation conditions.

Ageladine A is a pH-sensitive, membrane-permeable fluorescence dye. The change in the intra-cellular pH value is important for a range of diseases and in particular cancer calls exhibit a disturbed pH value. pH-dependent membrane-permeable fluorescence indicators are also suitable for monitoring endosomes, lysosomes and other organelles.

The natural substance ageladine A has a range of properties which make it an outstanding pH sensor: high level of membrane-permeability; only 15 minutes' incubation for cells; very wide range from pH 4 to pH 9; all experiments can be carried out with conventional laboratory equipment; stability; ageladine A is not toxic and can therefore also be used with sensitive cells (for example, PC12) and long incubation; ageladine A scarcely has any tendency to fade, even with relatively long incubation times. Ageladine A can be used in the following applications; fluorescence microscopy (living cells, tissue and even whole animals), frozen sections and optionally also flow cytometry.

The bioactive marine natural substance ageladine A (chemical formula C₁₀H₇N₅Br₂) is a pyrrole/imidazole alkaloid which can be isolated, for example, from sponges of the genus Agelas (cf. M. Fujita et al.: “Ageladine A: An Antiangiogenetic Matrixmetalloproteinase Inhibitor from Marine Sponge Agelas nakamurai”, J. Am. Chem. Soc. 2003, 125, 15700-15701 and Supporting Information S.I. 1-15). It is now also possible to completely synthesise ageladine A (cf. M. Meketa et al.: “Total Synthesis of Ageladine A, an Angiogenesis inhibitor from the Marine Sponge Agelas nakamurai” Org. Lett. 2006, 8, 7, 1443-1446; Publication by S. Shengule et al.: “Concise Total Synthesis of the Marine Natural Product Ageladine A”, Org. Lett. 2006, 8, 18, 4083-4084; and M. Mekata et al.: “A New Total Synthesis of the Zinc MatrixMetalloproteinase inhibitor Ageladine A Featuring a Biogenetically Patterned 6[pi]-2-Azatriene Electrocyclization”, Org. Lett, 2007, 9, 5, 853-855). Consequently, ageladine A is available to the public to an unlimited extent. Ageladine A has pronounced fluorescence in the green range after UV excitation. Furthermore, ageladine A is discussed in scientific literature with respect to various subjects such as, for example in: Bickmeyer, U.; Grube, A.; Klings, K. W.; Köck, M. Ageladine A, a pyrrole-imidazole alkaloid from marine sponges, is a pH sensitive membrane permeable dye, Biochem. Biophys. Res. Commun. 2008 373, 419-422. Erratum in: Biochem Biophys Res Commun. 2009 Jun. 12;383(4);519. Bickmeyer, U.; Heine, M.; Podbielski, I.; Münd, D.: Köck, M.; Karuso, P, Tracking of fast moving neuronal vesicles with ageladine A. Biochem Biophys Res Commun. 2010. 402, 489-494. Parks, S. K.; Chiche, J.; Pouyssegur, J. pH control mechanisms of tumor survival and growth. J Cell Physiol. 2011 228, 299-308. Webb, B. A.; M; Chimenti, M; Jacobsen, M. P; Barber, D. L.; Dysregulatad pH: a perfect storm for cancer progression. Nature Reviews Cancer 2011, 11, 671-677.

The person skilled in the art may adapt and carry out the present invention on the basis of information known from the prior art in relation to ageladine A without particular difficulties with respect to his specific requirements in the individual case. Thus, for example, DE 10 2007 034866 whose disclosure is hereby expressly incorporated by reference for the purposes of the present invention describes a method relating to how pH value measurements of solutions and pH value measurements within living cells, tissues and whole organisms as a medium can be carried out using ageladine A as a fluorescing pH value indicator. Thus, a fluorescence dyeing of cells and tissues is possible by incubating those media with ageladine A. Advantageously, it is possible to carry out an in-vivo or in-vitro incubation of cells, tissues or complete organisms as a medium in a solution which contains the fluorescing pH value indicator. As a result of such simple, rapid and biological dyeing, it is possible to mark according to DE 10 2007 034866, for example, acidic tissue portions (digestive organs) in living organisms and to readily identify them under the fluorescence microscope. Therefore, ageladine A can be used as an extremely intensive cell dye which simultaneously indicates pH value changes as a pH value indicator in a reproducible manner. Qualitative pH value indications and quantitative pH value measurements within living systems can be greatly simplified by using ageladine A.

The hydrogen concentration or the pH value is an extremely important parameter in biological and chemical/technical systems. Many chemical and biological reactions require precise control of the pH value for correct execution. In the context of optical measurements, there are used pH value indicators which involve dyes whose detectable optical properties, such as extinction (absorption) or fluorescence, also change with the change of the pH value. Consequently, pH value indicators indicate the current pH value of the solution by means of the colour and the colour intensity thereof. The greatest sensitivity of indicators to small changes of the pH value is present when the equilibrium constant (pKa) between the acidic and basic forms of the indicator is near the value of the medium to be examined, generally a solution.

The present invention makes use of the pH-value-dependent properties of ageladine A in an advantageous manner in order to assess the ability to live (viability) of biological samples, in particular biological samples in which a decreasing ability to live (viability) of the biological samples is associated with a change of the intra-cellular pH value, preferably with a reduction of the intra-cellular pH value, and particularly preferably with a reduction of the intra-cellular pH value in the acidic pH range.

In this instance, it is advantageous that ageladine A has a particularly high level of sensitivity of the emitted fluorescence intensity in the physiologically relevant range between pH 6 and pH 8. Consequently, ageladine A can be used as a pH value indicator particularly well for physiological pH value measurements in order to assess or establish the ability to live (viability) of biological samples. Advantageously, physiological samples (in-vitro) which are taken to assess or to establish the ability to live (viability) but also living cells, tissues and whole organisms (in-vivo) can be incubated with ageladine A as a fluorescing pH value indicator and can thereby be dyed by the fluorescence in the green range. This allows, by means of measurements of the pH value in-vivo and in-vitro in a wide range, the identification of non-physiological and therefore harmful pH value changes in the organism to be tested, in particular the identification of acidic tissues in organisms, as a result of the fluorescence properties of ageladine A, and consequently, as a result, also the assessment or establishment according to the invention of the ability to live (viability) of the organisms to be tested. The fluorescence in the green range increases substantially as the pH value decreases. The intensive dyes have been found to be stable over hours and days.

Ageladine A is surprisingly suitable in this instance in a very particularly advantageous manner for assessing or establishing the ability to live (viability) of such biological samples as ovaries of parasitic worms, preferably ovaries of helminthes, particularly preferably ovaries of the pig whipworm (Trichuris suis). Therefore, the invention provides a substantially simplified cost-effective, efficient and reliable method which is extremely advantageous for economic, ethical and practical reasons for establishing the viability in comparison with the in-vivo method of the prior art which carries out the establishment of the viability of ovaries of the pig whipworm in living pigs.

In another embodiment, therefore, the invention also relates to a kit for assessing the ability to live (viability) of one or more biological samples comprising ageladine A, preferably in the form of a stock solution, as a fluorescence dye for dyeing biological sample(s) to be tested, and particularly preferably adapted for use in conjunction with biological samples in an object carrier with recesses, in a microtitre plate having a large number of recesses or in a microfluidic chip.

In preferred embodiments, the kit according to the invention for assessing the ability to live (viability) of one or more biological samples may further comprise at least one of the following components and, in particularly preferred embodiments, is adapted for use in conjunction with biological samples in an object carrier with recesses, in a microtitre plate having a large number of recesses or in a microfluidic chip. In addition to ageladine A, the kit according to the invention may further comprise, for example, at least one of the following components: a) a cultures medium for incubating the biological sample(s) to be tested; b) a buffer medium; c) a washing medium which may optionally be buffered; d) a fluorescence calibration standard; e) a positive and/or negative control; f) optionally one or more object carriers with recesses and one or more glass covers; g) optionally one microtitre plate having a large number of recesses or a microfluidic chip; and h) optionally instructions for using the kit in individual tests, group tests and/or high-throughput tests.

Additional advantages and advantageous embodiments of the invention may be taken from the following examples and the claims. All the features set out in the description, the following examples and the claims may be inventively significant both individually and in any combination With each other.

The operation of the invention is now intended to be described below in greater detail with reference to examples, but without wishing to thereby limit the invention in terms of its scope. The scope of the invention is defined in the patent claims and is supported by the above, detailed description. The examples are used for additional explanation.

### EXAMPLES

### Establishing the Viability by Means of Ageladine A Using the Example of Eggs of the Whipworm

1) Introduction:

Basic principles: the alkaloid ageladine is uncharged in an alkaline medium whereas it is protonated in an acidic medium and is therefore present in a charged state (Bickmeyer at al. 2010 BBRC), in the charged state, ageladine is membrane-impermeable whereas it very rapidly crosses many cell membranes in the uncharged state. This is also substantially as a result of the double-bromating of the molecule which substantially increases the membrane accessibility in similar marine alkaloids (Bickmeyer et al. 2004, Toxicon). The eggs of the whipworm normally absorb dyes very poorly but the absorption of ageladine is very rapid owing to the bromating. Ageladine fluoresces in an increased manner with a decreasing pH (Bickmeyer et al. 2008 BBRC). Eggs whose oxygen and/or energy supply decreases for any reasons slow down their transport processes and cannot regulate the pH value well any more and become acidic. That acidification is responded to with a very high level of probability by ageladine with an increase in the fluorescence. In this manner, eggs whose metabolism is disrupted or which already die can be clearly distinguished from healthy eggs.

Therefore, this test is extremely sensitive in distinguishing between healthy eggs and eggs which have already been damaged. Completely dead eggs should absorb the dye very well as a result of non-present protection processes and fluoresce powerfully.

A provision of the optical control for establishing the quality of the eggs as being viable versus embryonated or for the delimitation thereof can be drawn up correctly in the respective case on the basis of relevant criteria and on the basis of the biology of Trichuris suis for the technical (for example, pharmaceutical) and/or legal (for example, legal authorisation) requirements. Similarly, the establishment or definition of a threshold value can be carried out: light intensity in comparison with the background (noise). A computer-controlled evaluation is possible. A correlation between viable ISO (motility test, pig infectivity test and slip test according to Abromelt) and the ageladine-dyed eggs may also be produced, as well as the establishment of a clear definition: embryonated as distinct from viable.

2) Material and Methods:

2.1 Reference Companies:
Alfred-Wegener-Institut. für Polar- und Meeresforschung; (AWI), D-27570
Eppendorf-Netheler-Hinz GmbH, D-22331 Hamburg
Gibco BRL Life Technologies GmbH, D-76339 Eggenstein
Haereus Instruments GmbH, D-63450 Hanau
IKA®-Werke GmbH & Co, KG, D-79219 Staufen
Leica Mikrosysteme Vertrieb GmbH, D-64625 Bensheim
Merck KG, D-84271 Darmstadt
Nunc Thermo Electron LED GmbH, D-63505 Langenselbold
Ovamed GmbH, D-22885 Barsbüttel
Sigma-Aldrich Chemie GmbH, D-82039 Deisenhofen

2.2 Apparatuses:
Tubes, pipette tips, etc. Eppendorf/Nunc
Pipettes: 1-10 μl, 100-1000 μl Eppendorf
Multi-Photon-Microscope SP5 MP Leica
Objective HC PL FLUOTAR 10.0× 0.30 DRY Leica
Biofuge 13 Haereus
LabDancer IKA

2.3 Chemicals:
Ageladine A (AWI): bromated pyrrole/imidazole alkaloid)
All other chemicals not set out were obtained from Merck.

2.4 Solutions, Buffers and Media, Etc:
DPBS (Ca/Mg) pH 7.4 (Gibco)

2.5 Test Animals:

Embryonated eggs of Trichuris suis (TSO) according to specification of Ovamed GmbH. The nematode eggs are stored in the refrigerator at from 4 to 8° C. in a phosphate buffer at pH 5 with addition of potassium sorbate. For the tests, the TSO were washed several times in 1×DPBS (re-buffering) and adjusted to a concentration of 1000 TSO/ml.

2.6 Histology:

2.6.1 Fluorescence:

| | Excitation | Bandpass filter |
|---|---|---|
| Substance | (Wavelength light in nm) | (Wavelength in nm) |
| Ageladine A | 370 | 410-480 |

2.6.2 Establishment of Ageladine A:

Ageladine stock solution: 10 mM in MeOH (90%); 1 μl of ageladine was combined with 1000 μl of TSO suspension (see 2.5) in 15 ml tubes. The contents were mixed by gentle shaking and incubated for 10 minutes at ambient temperature in the dark.

3) Method:

The ISO were washed three times in DPBS after being dyed with Ageladine A (see 2.6.2). 10 μl of the samples were placed on object carriers with recesses and covered with glass covers. The object carriers were observed under the microscope Leica SP5 MP with and without UV excitation.

4) Results:

After incubation with ageladine A, a small percentage of the TOS observed under the microscope with excitation at a wavelength of 370 nm in the range between 410 and 480 nm exhibited fluorescence. Nevertheless, a quotient which fixes the ratio of fluorescing to non-fluorescing ISO was not determined. It was not possible to observe any auto-fluorescence with UV excitation at the wavelength mentioned (see illustration 1).

Illustration 1: Ageladine dyes eggs of the whipworm (see transmitted-light image). There are shown three microscopic images (ten-fold enlargement) of randomly selected, identical and embryonated eggs of the whipworm Trichuris suis: it is possible to see larval structures surrounded by an oval egg case. The TSO on the images on the left and at the centre with green fluorescence after excitation with light of the wavelength 370 nm and with a different background; the image on the right without UV excitation. TSO are on average 25 μm wide and 70 μm long; a scale is not shown.

Furthermore, selected eggs in limited numbers were evaluated under the transmitted-light microscope in terms of quality according to morphological criteria and designated “good” (embryonated, viable) or “poor” (non-embryonated, detectable large vacuoles, digestion) and located on the computer monitor (blind test establishment). The illustration 2 shows an example: four of a total of fourteen TSO received the qualify designation “good”. Defective eggs were graphically marked with white arrows. The control of the same TSO with UV excitation shows a high level of correlation between the marked eggs and the fluorescing eggs. One additional egg exhibits dyeing as a result of ageladine A (red arrow).

Illustration 2: Ageladine dyes in particular eggs with defects (optical blind test):

Illustration 2 shows microscopic images (10×) of identical eggs of Trichuris suis (on the left with UV excitation, on the right without): white arrows on both images indicate TSO which has been previously classified as being non-embryonated or as being defective according to morphological criteria. In comparison, a fluorescing egg (with red arrow) which was designated “good” according to optical control. A scale is not shown.

1. A method for assessing the viability of one or more biological samples comprising:
a) the provision of the biological sample(s) to be examined;
b) the incubation of the biological sample(s), provided at a) with ageladine A;
c) the ultraviolet excitation of the incubated biological sample obtained at b) with UV light of a wavelength of 370 nm; and
d) the detection of fluorescing and non-fluorescing portions of the biological sample(s) excited with ultraviolet according to c) in the range between 410 and 480 nm.

2. The method for assessing the viability of biological samples according to claim 1, wherein the biological samples resulted from biological preparations which are used in a viable form to produce medicaments and/or therapeutic agents.

3. The method for assessing the viability of biological samples according to claim 1, wherein the biological sample is selected from the group consisting of (lower) organisms, in particular microorganisms, parasitic and non-parasitic organisms, cells, individual cells or cell colonies, tissues, organelles, sperm, egg cells and ovaries, wherein the biological sample is preferably selected from the group consisting of parasitic and non-parasitic organisms and ovaries of such parasitic and non-parasitic organisms.

4. The method for assessing the viability of biological samples according to claim 1, wherein there are selected as biological samples ovaries of parasitic and/or non-parasitic worms, preferably ovaries of parasitic and/or non-parasitic worms which are used in a viable form to produce medicaments and/or therapeutic agents.

5. The method for assessing the viability of biological samples according to claim 1, wherein there are selected as biological samples ovaries of parasitic worms, preferably ovaries of helminthes, particularly preferably ovaries of the pig whipworm.

6. The method for assessing the viability of biological samples according to claim 1, wherein a decreasing viability of the biological samples is associated with a change of the intra-cellular pH value, preferably with a reduction of the intra-cellular pH value, and particularly preferably with a reduction of the intra-cellular pH value in the acidic pH range.

7. The process of assessing and/or establishing the viability of one or more biological samples by means of fluorescence, comprising: using ageladine A in order to assess and/or to establish the viability of one or more biological sample(s) by means of fluorescence.

8. A kit for assessing the viability of one or more biological samples comprising ageladine A, as a fluorescence dye for dyeing biological sample(s) to be tested.

9. The Kit for assessing the viability of one or more biological samples according to claim 8, further comprising at least one of the following components, preferably adapted for use in conjunction with biological samples in an object carrier with recesses, in a microtitre plate having a large number of recesses or in a microfluidic chip:
a) a culture medium for incubating the biological sample(s) to be tested;
b) a buffer medium;
c) a washing medium which may optionally be buffered;
d) a fluorescence calibration standard;
e) a positive and/or negative control;
f) optionally one or more object carriers with recesses and one or more glass covers;
g) optionally a microtitre plate having a large number of recesses or a microfluidic chip;
h) optionally instructions for using the kit in individual tests, group tests and/or high-throughput tests.

10. The process of assessing and/or establishing the viability of one or more biological samples by means of fluorescence according to claim 7, wherein biological samples can be intracellularly dyed by conventional fluorescence dyes other than ageladine A only with difficulty.
